(19) United States
(12) Patent Application Publication
(10) Pub. No.: US 2023/0132971 A1
(43) Pub. Date: May 4, 2023

(54) BIODEGRADABLE JOINT IMPLANTS, ITS PREPARATION METHOD AND A JOINT REPLACEMENT METHOD

(71) Applicant: Chang Gung University, Taoyuan City (TW)

(72) Inventors: Shih-Jung LIU, Taoyuan City (TW); Ying-Chao CHOU, Taoyuan City (TW); Chao-Lin CHEN, Taoyuan City (TW); Zhe-Pei WANG, Taoyuan City (TW)

(21) Appl. No.: 17/706,003

(22) Filed: Mar. 28, 2022

(30) Foreign Application Priority Data
Nov. 4, 2021 (TW) 110141198

(51) Int. Cl. A61F 2/42

(52) U.S. Cl. CPC A61F 2/4241; A61F 2002/30062

(57) ABSTRACT

A biodegradable joint implant comprises a joint scaffold and a film layer coated on the surface of the joint scaffold. The joint scaffold comprises a first anchor fixing, a second fixing and a flexible spacer, and the first anchor fixing and the second anchor fixing are respectively axially connected to opposite sides of the flexible spacer. The joint implant is made of biodegradable material, and the film layer contains at least one substance that can induce tissue growth. A method for preparing the joint implant and a joint replacement method using the joint implant are also provided.

### CROSS-REFERENCE TO THE RELATED APPLICATIONS

This application is based upon and claims priority to Taiwan Patent Application No. 110141198, filed on Nov. 4, 2021; the disclosure of which is incorporated herein by reference in its entirety.

### FIELD OF THE PRESENT DISCLOSURE

The present disclosure relates to a biodegradable joint implant, the method for preparing the joint implant and its use.

### BACKGROUND

Rheumatoid Arthritis (RA) is an autoimmune disease of unknown etiology that affects various tissues and organs throughout the body. At present, some studies reported that special genetic background or acquired environmental infection may contribute to the disease, but the exact cause has not yet been found. RA affects about 0.5 to 1% of adults in developed countries and affects about 5 to 50 out of 100,000 people each year. The main symptom of RA is chronic inflammatory synovitis, which results in erosion of cartilage. Early symptoms of RA can cause joint pain and swelling. If not treated in time, it will cause severe joint deformation and deformity. Patients often suffer from various symptoms due to inflammation, such as pain, impaired mobility, and even disability. Once suffering from RA, the patient will suffer from pain for life, and various symptoms will seriously affect the ability to live and move.

If RA does not have proper treatment within a few years of its onset, the joints will be destroyed. For joints that have been destroyed, surgical methods can be used to reconstruct their functions, such as artificial joint replacements to repair damaged and deformed joint tissue.

Silicone artificial joint replacement is currently most widely used method to treat the destruction of the metacarpophalangeal joint (MCP) caused by RA (Swanson A B., J Bone Joint Surg., 1972). The method is to remove the faulty phalangeal tissue of the fingers, and use high-strength artificial silicone to act as the interphalangeal cartilage to give space for the knuckles to move. However, after a long-term follow-up of more than 10 years, it was found that the silicone artificial joints were break and wear due to the aging of the material. See, e.g., I. A. Trail et al., The Bone & Joint Journal., 2004. At this time, it must be replaced and repaired by surgery, thereby increasing the suffering of the patient. Therefore, it is an urgent problem to develop better artificial joints, especially artificial finger joints or toe joints, to reduce or relieve the suffering of patients.

We uses a biodegradable joint implant as a temporary support frame, which is coated with a film layer containing tissue growth factors and other medicines, and the growth factors and other medicines are used to induce human tissue growth and ease the conditions. The joint implant will gradually degrade and be replaced by human tissue to form a human joint-like to improve the shortcomings of conventional silicone finger joints.

### SUMMARY

The present disclosure provides a biodegradable joint implant. The joint implant comprises a joint scaffold and a film layer, and the film layer is coated on the surface of the joint scaffold, wherein the scaffold comprises a first anchor fixing, a second anchor fixing and a flexible spacer, and the first anchor fixing and the second anchor fixing are respectively axially connected to opposite sides of the flexible spacer. The joint implant is made of biodegradable material, and the film layer comprises at least one substance for inducing tissue growth.

The present disclosure also provides a method for preparing a joint implant. The method comprises preparing a joint scaffold with a biodegradable material, wherein the joint scaffold comprises a first anchor fixing, a second anchor fixing and a flexible spacer, and the first anchor fixing and the second anchor fixing are respectively axially connected to opposite sides of the flexible spacer. Then a film layer is then prepared on the joint scaffold.

The present disclosure also provides a joint replacement method. The method comprises replacing the injured joint with the joint implant prepared by the method as described herein by performing joint replacement surgery.

The present disclosure also provides a method for treating joint injury caused by rheumatoid arthritis. The method comprises replacing the injured joint with the joint implant prepared by the method as described herein by performing joint replacement surgery.

### BRIEF DESCRIPTION OF THE DRAWINGS

Further features and advantages will become apparent from the following and more particular description of the preferred embodiments of the disclosure, as illustrated in the accompanying drawings, and in which like referenced characters generally refer to the same parts or elements throughout the views, and in which:

FIG. 1A is a stereogram of an exemplary joint implant, and FIG. 1B is a side view of the joint implant.

FIG. 2A is a stereogram of an exemplary joint implant, and FIG. 2B is a side view of the joint implant.

FIG. 3A is a stereogram of an exemplary joint implant, and FIG. 3B is a side view of the joint implant.

FIG. 4A is a stereogram of an exemplary joint implant, and FIG. 4B is a side view of the joint implant.

FIG. 5 is a SEM image of electrospinning fiber film.

FIGS. 6A to 6C show the hydrophilic and hydrophobic properties of the joint implants coated with the electrospinning fiber film having no drugs and having two drugs detected by using an angle measuring instrument for horizontal imaging.

FIG. 7A shows the in vitro daily releases of drug ingredients in the film layer, and FIG. 7B shows the in vitro cumulative releases of drug ingredients in the film layer.

FIG. 8 shows a degradation curve of PCL scaffold prepared by the method as described herein.

### DETAILED DESCRIPTION

At the outset, it is to be understood that this disclosure is not limited to particularly exemplified materials, architectures, routines, methods or structures as such may vary. Thus, although a number of such options, similar or equivalent to those described herein, can be used in the practice or embodiments of this disclosure, the preferred materials and methods are described herein.

It is also to be understood that the terminology used herein is for the purpose of describing particular embodiments of this disclosure only and is not intended to be limiting.

The detailed description set forth below in connection with the appended drawings is intended as a description of exemplary embodiments of the present disclosure and is not intended to represent the only exemplary embodiments in which the present disclosure can be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the specification. It will be apparent to those skilled in the art that the exemplary embodiments of the specification may be practiced without these specific details. In some instances, well known structures and devices are shown in block diagram form in order to avoid obscuring the novelty of the exemplary embodiments presented herein.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

For purposes of convenience and clarity only, directional terms, such as top, bottom, left, right, up, down, over, above, below, beneath, rear, back, and front, may be used with respect to the accompanying drawings. These and similar directional terms should not be construed to limit the scope of the disclosure in any manner.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

Unless defined otherwise, all technical and scientific terms used herein have the same meaning as commonly understood by one having ordinary skill in the art to which the disclosure pertains. Moreover, as used in this specification and the appended claims, the singular forms “a”, “an” and “the” include plural referents unless the content clearly dictates otherwise.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, a “therapeutically effective amount” or “effective amount” of a medicament is that amount of medicament that is sufficient to provide a beneficial effect to the subject to which the medicament is administered.

As used herein, a “patient” or “subject” may be a human or non-human mammal or an avian. Non-human mammals include, for example, livestock and pets, such as ovine, bovine, porcine, canine, feline and murine mammals. In certain embodiments, the subject is human.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. This applies regardless of the breadth of the range.

### Joint Implants

The present disclosure provides a biodegradable joint implant prepared by a biocompatible material. The joint implant comprises a joint scaffold and a film layer, wherein the scaffold comprises a first anchor fixing, a second anchor fixing and a flexible spacer, and the first anchor fixing and the second anchor fixing are respectively axially connected to opposite sides of the flexible spacer.

The flexible spacer further comprises a longitudinal first side wall and a longitudinal second side wall and the first anchor fixing extends laterally outward from the outer side of the first side wall, and the second anchor fixing extends laterally outward from the outer side of the second side wall, and the first side wall and the second side wall are connected by one or more lateral connecting parts. The configuration of the connecting part is not limited as long as the first side wall and the second side wall can be firmly connected. In a preferred embodiment, the connecting part can be in the shape of a flat plate, a square column, a cylinder and the like. In another preferred embodiment, the connecting part is hinge-shaped.

In an embodiment, the flexible spacer comprises a longitudinal first side wall and a longitudinal second side wall, and the both ends of the connecting part are laterally connected to the inside central portions of the first side wall and the second side wall respectively to form a H-shaped flexible spacer. The first anchor fixing and the second anchor fixing extend laterally outward from the first side wall and the second side wall respectively.

In an embodiment, the flexible spacer comprises a longitudinal first side wall and a longitudinal second side wall, and both ends of the connecting part are laterally connected to the inside bottoms or tops of the first side wall and the second side wall respectively to form an inverted U-shaped or U-shaped flexible spacer. The first anchor fixing and the second anchor fixing extend laterally outward from the first side wall and the second side wall respectively.

In an embodiment, the flexible spacer comprises a longitudinal first side wall and a longitudinal second side wall, and the connecting part is hinge-shaped. That is, two connecting parts are laterally connected to the bottom and top of the first side wall respectively, and each end of the two connecting parts away from the first side wall has a hole. The top and bottom of the second side wall have shaft devices corresponding to the holes on the two connecting parts. The two shaft devices of the second side wall are respectively mounted into the corresponding holes on the two connecting parts to form a flexible spacer with a hinge configuration. The first anchor fixing and the second anchor fixing extend laterally outward from the first side wall and the second side wall respectively.

The shapes, lengths and sizes of the first anchor fixing and the second anchor fixing in the joint implant described herein are not limited, and can be estimated and determined by the surgeon based on the joint and bone structures of the patient's target joint or other factors. In a preferred embodiment, the first anchor fixing and/or the second anchor fixing are conical-shaped, truncated cone-shaped, pyramid-shaped, truncated pyramid-shaped, cylindrical-shaped or square column-shaped. In a preferred embodiment, the first anchor fixing and the second anchor fixing are conical-shaped.

In the joint implant of the present disclosure, the first anchor fixing and the second anchor fixing extend laterally outward from the first side wall and the second side wall respectively, wherein the axis of the extending direction from the first anchor to the second anchor (that is, the axial section of the joint implant of the present disclosure) may be a straight line or an arc line, and can be estimated and determined by the surgeon based on the joint and bone structures of the patient's target joint or other factors. In a preferred embodiment, the axis of the extending direction from the first anchor to the second anchor is an arc line.

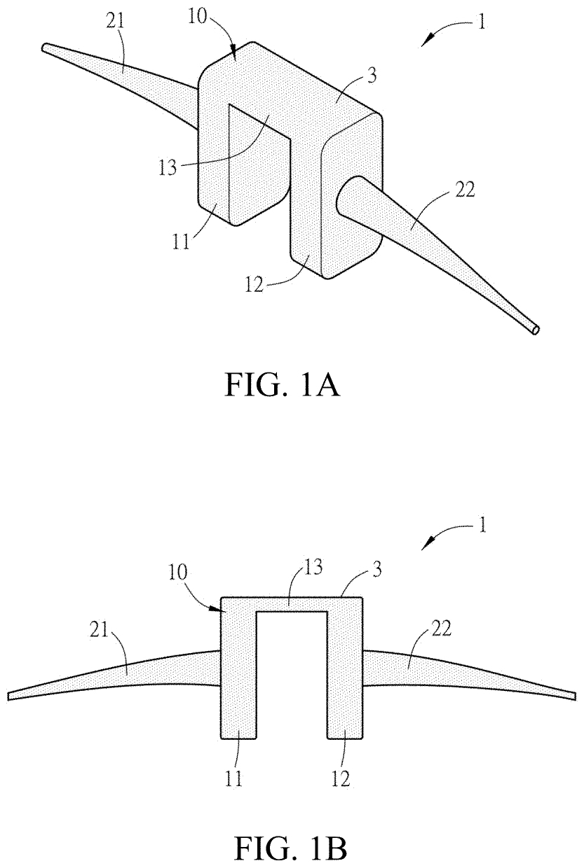

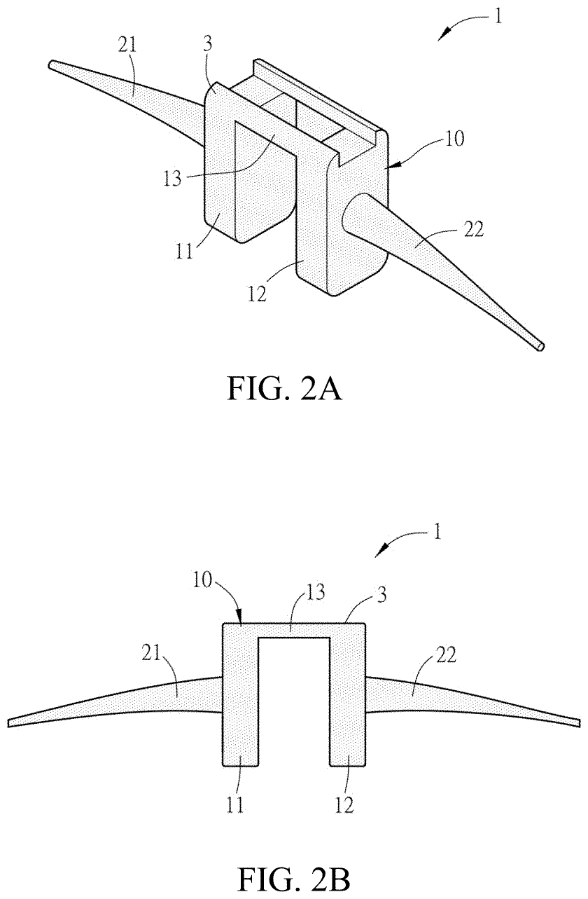

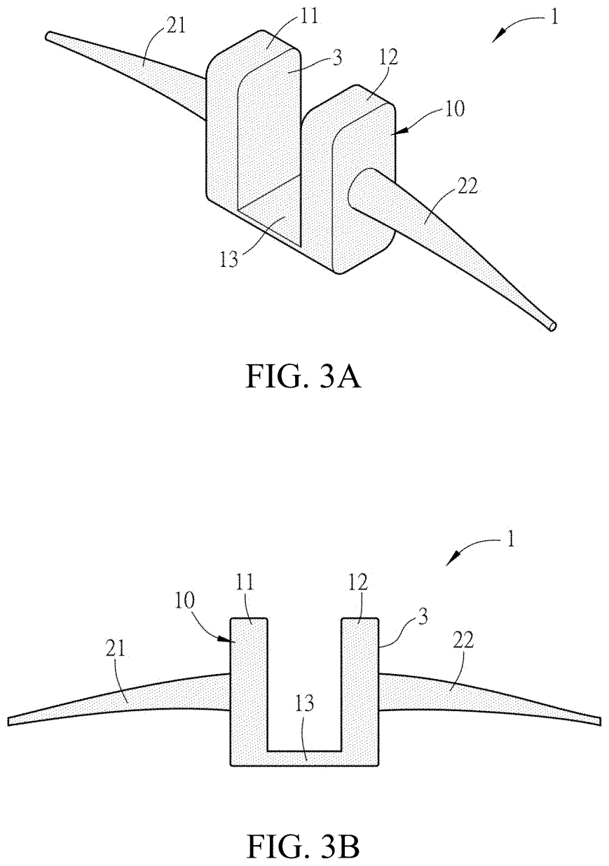

Referring to the embodiment of FIGS. 1A and 1B, the joint implant disclosed herein comprises a joint scaffold 1 and a film layer 3, wherein the joint scaffold comprises a first anchor fixing 21, a second anchor fixing 22 and a flexible spacer 10. The flexible spacer 10 further comprises a longitudinal first side wall 11 and a longitudinal second side wall 12, and the first side wall 11 and the second side wall 12 are connected by one or more connecting parts 13. The first anchor fixing 21 extends laterally outward from the outer side of the first side wall 11, and the second anchor fixing 22 extends laterally outward from the outer side of the second side wall 12, wherein the axis of the extending direction from the first anchor 21 to the second anchor 22 is an arc line. In other embodiment, the joint implant disclosed herein is shown in FIGS. 2A and 2B. In other embodiment, the joint implant disclosed herein is shown in FIGS. 3A and 3B.

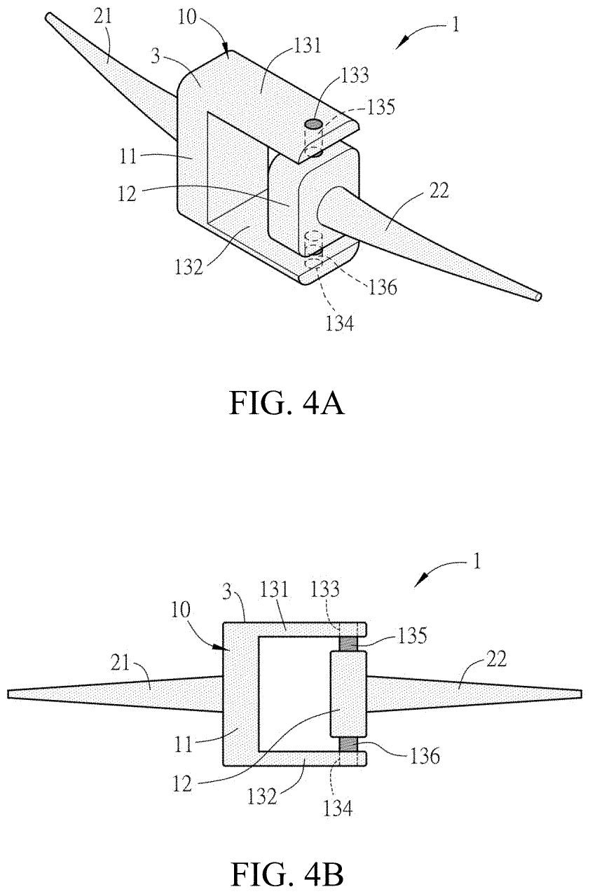

Referring to another embodiment of FIGS. 4A and 4B, the joint implant disclosed herein comprises a joint scaffold 1 and a film layer 3, wherein the joint scaffold comprises a first anchor fixing 21, a second anchor fixing 22 and a flexible spacer 10. The flexible spacer 10 further comprises a longitudinal first side wall 11 and a longitudinal second side wall 12 and two connecting parts 131 and 132. The two connecting parts are laterally connected to the bottom and top of the first side wall 11 respectively. The ends of the two connecting parts away from the first side wall 11 have holes 133 and 134, and the top and bottom of the second side wall 12 have shaft devices 135 and 136 corresponding to the two holes on the two connecting parts. The two shaft devices 135 and 136 are respectively mounted into the corresponding holes 133 and 134 on the two connecting parts to form a flexible spacer with a hinge configuration. The first anchor fixing 21 extends laterally outward from the outer side of the first side wall 11, and the second anchor fixing 22 extends laterally outward from the outer side of the second side wall 12, wherein the axis of the extending direction from the first anchor 21 to the second anchor 22 is an arc line.

The joint implant disclosed herein can be applied to joints such as in palms, wrists, elbows, and soles, and is suitable for patients of all ages. Therefore, the size of each component of the joint implant of the present disclosure can be made according to the size of the joint to be reconstructed, and is not limited.

In an embodiment, the joint implant of the present disclosure is used for joint reconstruction in the fingers. In this case, the length of the flexible spacer can be 10-30 mm, preferably 10-25 mm, and more preferably 10-20 mm. In an embodiment, the size of the flexible spacer can be 10, 15, 20, 25 mm or other suitable sizes. The length of the first anchor can be identical with or different from that of the second anchor. The surgeon may consider factors such as the structure of patient's joint to be reconstructed, the surgical method, and the structural support of the joint implant of the present disclosure before cutting said joint implant. In an embodiment, the lengths of the first anchor fixing and the second anchor fixing are respectively 20-40 mm, preferably 20-35 mm and more preferably 20-30 mm. In an embodiment, the sizes of the first anchor fixing and the second anchor fixing can be 10, 15, 20, 25 mm or other suitable sizes, respectively. In other embodiment, the joint implant of the present disclosure can be customized according to the size of the patient's joint to be reconstructed. For example, the joint implant can be quickly manufactured and provided according to the size of the patient's joint to be reconstructed by using the 3D bioprinting technology described herein (as detailed below).

The joint implant of the present disclosure has a non-smooth surface to facilitate the attachment and growth of tissue cells. In an embodiment, the surface of the joint implant has a plurality of tiny holes in and/or penetrating it or has a micro-net-like fiber structure. The plurality of tiny holes or the micro-net-like fiber structure can promote the attachment and growth of fibrous tissue cells, and guide and control the growth of fibrous tissue into the joint scaffold and proliferate tissue. Therefore, when the scaffold is gradually degraded and absorbed, fibrous tissue will gradually replace the joint scaffold, and finally a functional fibrous joint without the joint implant is formed.

### Biodegradable Materials

As used herein, the term “biodegradable material” refers to a material that can be degraded or enzymatically degraded in the organism, and the generated small molecular substances can be absorbed by the organism and excreted from the body. Generally, biodegradable materials usually have the advantages of good biocompatibility, controllable degradation rate, sufficient mechanical strength, non-toxic and metabolizable, etc., so they are suitable as materials that are not removed after being implanted in the body, such as surgical sutures or drug delivery carrier, and even as scaffolds for tissue engineering. See, e.g., Yajie Zhong, et al., Advanced Industrial and Engineering Polymer Research, pp. 27-35, 2020. In an embodiment, the joint scaffold of the present disclosure is made by biodegradable materials.

In an embodiment, the biodegradable materials used herein can be selected from a group consisting of poly(lactide-co-glycolide) (PLGA), polycaprolactone (PCL), and any combination thereof. In an embodiments, the biodegradable materials used herein is PLGA. In an embodiments, the biodegradable materials used herein is PCL.

PLGA is copolymerized by a cyclic dimer of glycolic acid (GA) and lactic acid (LA), and has been approved by the U.S. Food and Drug Administration (FDA) for clinical applications.

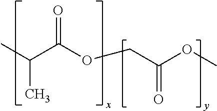

Because of its good biocompatibility, biodegradability and minimal toxicity, PLGA has become one of the most commonly used synthetic materials for preparing fiber scaffolds in tissue engineering. Among many biomaterials, biodegradable PLGA has shown great potential as a drug delivery carrier and as scaffolds for tissue engineering. See, e.g., Hirenkumar K. et al., Bioinspired Polymers, pp. 1377-1397, 2011.

PCL is a semi-crystalline, hydrophobic and biodegradable polyester with good thermal processing properties, that is, the glass transition temperature is −60° C. and the melting point is quite low (55-64° C.). Therefore, PCL has been approved by the FDA as a polymer suitable for use in the human body. Because of its cost-effectiveness, high toughness and good biocompatibility, it is often used as the main material of drug delivery systems. Under physiological conditions, PCL degrades more slowly than other biodegradable polyesters. This property can control the release of drugs in target tissues for a period of time. PCL has low hardness, flexible, high ductility, and good mechanical properties, especially in elastic recovery and wear resistance, and also has good weather resistance. With various advantages, PCL is also often used in bioprinting to manufacture many scaffolds used in the body. See, e.g., Yajie Zhong, et al., Advanced Industrial and Engineering Polymer Research, pp. 27-35, 2020.

### Medicines

The biodegradable joint implant provided in the present disclosure comprises a film layer comprising a therapeutically effective amount of at least one medicine, which can be useful for the purposes of promoting tissue growth, reducing pain, preventing infection, promoting absorption and the like. In an embodiment, the film layer of the joint implant provided in the present disclosure comprises a therapeutically effective amount of at least one medicine selected from a group consisting of tissue growth factor-related drugs, anti-inflammatory drugs, antibiotics, antimicrobials, pain relievers, wound healing agents, anesthetics, and combinations thereof.

In an embodiment, the film layer can provide sustained or continuous release of the drug(s) contained therein. The term “sustained release” or “continuous release” is used in its conventional sense to refer to a drug formulation that provides for gradual release of a drug over an extended period of time.

In an embodiment, a therapeutically effective amount of the medicine comprised in the film layer is a tissue growth factor-related drug. The tissue growth factor-related drugs can induce or promote the growth of human tissues, so that the tissue can be formed into joint-like tissue in situ to replace the gradually degenerated joint implant. The specific example of the tissue growth factor useful in the present disclosure comprises bone morphogenetic protein 2 (BMP-2). BMP-2 is a transforming growth factor beta (TGF-β). BMP-2 can be used in various treatments, such as, bone defects, nonunion fractures, spinal fusion, osteoporosis and root canal surgery. BMP-2 is an osteoinductive growth factor approved by U.S. Food and Drug Administration (FDA) for use as a bone graft substitute. See, e.g., Di Chen et al., Growth Factors, pp. 233-241, 2004.

The specific example of the useful tissue growth factor also comprises a connective tissue growth factor (CTGF). CTGF is a heparin-binding protein associated with the extracellular matrix (ECM) and binds directly to integrins. CTGF is synthesized by fibroblasts and stimulates the proliferation and chemotaxis of these cells. After injury, CTGF expression is increased, and CTGF is involved in granulation tissue formation, re-epithelialization, and matrix formation and reconstruction. In vitro experiments showed that CTGF promotes endothelial cell proliferation, migration, survival and adhesion during angiogenesis, and it was also confirmed that CTGF promotes vascular endothelial growth and is required for re-epithelialization in wound healing by promoting cell migration. See, e.g., Stephan Barrientos, et al., Wound Repair and Regeneration, pp. 585-601, 2008. In a specific embodiment, the drug is BMP-2 or CTGF or a combination of BMP-2 and CTGF.

In a specific embodiment, in addition to the above, the film layer may further comprise a therapeutically effective amount of the medicines selected from a group consisting of anti-inflammatory drugs, antibiotics, pain relievers or anesthetics, and combinations thereof. The examples of the anti-inflammatory drugs include, but are not limited to Adalimumab, Certolizumab, Etanercept, Golimumab, Abatacept, Tocilizumab, Rituximab, Infliximab and combinations thereof.

The examples of antibiotics include, but are not limited to Vancomycin, Teicoplanin, Ceftazidime, Gentamicin, Mezlocillin, Cloxacillin, Meticillin, Cephalothin, Lincomycin, Polymyxin E, Bacitracin, Fusidic Acid and combinations thereof.

The examples of pain relievers or anesthetics include, but are not limited to acetaminophen, Ketorolec, clonidine, benzodiazepine, lidocaine, tramadol, carbamazepine, meperidine, zaleplon, trimipramine maleate, buprenorphine, nalbuphine, pentazocain, fentanyl, propoxyphene, hydromorphone, methadone, morphine, levorphanol, hydrocodone and combinations thereof.

A medical doctor, e.g., surgeon or veterinarian, having ordinary skill in the art may readily determine the amounts of medicines comprised in the film layer of the present disclosure. For example, the surgeon or veterinarian could estimate doses of the medicines contented in the film layer according to the time required to achieve the therapeutic effect, the size of the lesion, and the scope of the operation.

### Use

The joint implant of the present disclosure can be used to replace the damaged and deformed joint of the patient. After replacing the damaged and deformed joint by the joint implant of the present disclosure, the flexible spacer of the joint scaffold is used as the joint body, and the first anchor fixing and the second anchor fixing extending from opposite sides of the flexible spacer are respectively implanted into the medullary cavity of the bones on both sides of the joint. The medicines contained in the film layer of the joint implant of the present disclosure can induce human tissue growth, so that the joint implant gradually degrades and is replaced by new human tissue, forming a joint-like structure to improve the patient's prognosis or physical appearance.

The joint implant of the present disclosure can be applied to joint damage and deformation caused by various reasons, including joint damage and deformation caused by various diseases. The diseases that can cause joint damage and deformation include rheumatoid arthritis, gout, arthritis, degenerative arthritis, psoriatic arthritis, etc. Long-term chronic inflammation in the joints leads to irreversible symptoms, such as, swelling, pain, bone hyperplasia, and stiffness in the joints, which eventually lead to joint damage and deformation. In an embodiment, the joint implant provided by the present disclosure can be used to treat joint damage and deformation caused by rheumatoid arthritis or gout.

The joint implant of the present disclosure can also be applied to joint damage and deformation caused by trauma, such as non-self-reparable joint damage and deformation caused by accident. The joint implant of the present disclosure can also be applied to postural reconstruction, such as postural deformation caused by recovery from illness or trauma, especially bone deformity caused by joint damage and deformation, for example, deformation of the palm due to long-term rheumatoid arthritis or gout.

The joint implant of the present disclosure can be applied to the reconstruction of unsupported or weakly supported joints, such as the joints of the palms, wrists, elbows, and soles of feet, preferably the joints of the palms and soles of feet, more preferably the joints of the fingers and the toes, and most preferably the joints of the fingers.

### EXAMPLES

### Materials and Methods

1. Printing Technology in Bioengineering

Three-dimensional (3D) bioprinting is an emerging technology for creating biomimetic tissues and organs, and forms complex and functional biological 3D structures by depositing cell-loaded biopolymers. The main three 3D bioprinting techniques including extrusion bioprinting, inkjet bioprinting, and Laser-based bioprinting have been proven suitable for 3D bioprinting. See, e.g., Wei Zhu et al., Current Opinion in Biotechnology, pp. 103-112, 2016. Partial polymer solutions or high-density aggregates are 3D bioprinted by extrusion to apply continuous force, printing uninterrupted cylindrical lines. 3D biomanufacturing technology can create functional 3D structures, which can contain one to several biological materials and cell types to simulate the natural microenvironment and biological composition, and are used in medical fields such as organ transplantation and organ regeneration. See, e.g., Christian Mandrycky et al., Biotechnology Advances, pp. 422-434, 2016.

2. Electrospinning Technology

Electrospinning is a widely used nanofabrication technology, which can directly and quickly spin polymers into nanofibers. The principle of electrospinning technology is to apply a high voltage to the liquid to generate static electricity, and the repulsive force between the charges will cancel out the surface tension of the liquid, so that the droplet is elongated. Increasing the electric field beyond a critical value, the repulsive electrostatic force uses this critical value to overcome the surface tension. The charged jet of fluid is ejected from the tip of the Taylor cone, so that the droplet is elongated, which allows the jet to become very long and thin. Meanwhile, the solvent is evaporated to leave a charged polymer fiber. Finally, the filaments form a very stable fibrous structure with a diameter of microns or nanometers on a grounded collector plate.

Electrospinning films can be used in wound dressings, biodelivery systems, controlled drug release, and tissue engineering scaffolds. It has found that more than fifty different polymers have been successfully electrospun into ultra fine fibers with diameters ranging from <3 nm to over 1 μm. See, e.g., Zheng-MingHuang, et al., Composites Science and Technology, Pages 2223-2253, 2003. Nanofibers prepared by electrospinning technology show advantages in the field of drug delivery due to their high surface area-to-volume ratio and interconnected pores in the fibers, which can ensure higher dissolution rates and higher absorption rates of therapeutic drugs. Furthermore, by adjusting the relevant nanofiber properties, such as fiber diameter, porosity, and drug binding mechanism, the drug release rate can be regulated for various applications.

### Example 1: Preparation of the Joint Implant Materials

The polymer material used in the preparation of joint scaffolds for joint implants by 3D printers is polycaprolactone (PCL) (commercially available from Sigma-Aldrich), with an average molecular weight Mn=80,000, non-toxic and medical grade Particles, and the degradation time of about 2 years, and good mechanical strength and toughness. The solvent used is dichloromethane (commercially available from Sigma-Aldrich). Dichloromethane is more stable than its congeners, such as chloroform and carbon tetrachloride, under normal temperature and moisture-free environment. The polymer material was prepared with a ratio of PCL to DCM of 2500 mg to 3.5 ml, and then placed in a 20 ml syringe. After standing for 6 hours to fully mix the solution, the polymer material can be used for 3D printing.

### Example 2: Preparation of Joint Implant

The joint implant was 3D printed by using a plunger extrusion 3D printer (U-Maker 3D printer, STEPS SOFTWARE INC., Taiwan). The feeding equipment was changed to a solvent extrusion device equipped with a 20 ml syringe and a dispensing needle. The polymer solution is prepared by dissolving polymer particles or powder in an organic solvent for printing.

### Example 3: Preparation of Drug Films by Electrospinning

The biodegradable polymer, poly(Lactide-co-Glycolide PLGA) (Resomer RG 503, from EVONIK Industries AG), was used for preparing electrospinning fiber films, and the solvent is 1,1,1,3,3,3-hexafluoro-2-propanol (HFIP) (Sigma).

The films were uniaxial electrospinning films containing the antibiotics Teicoplanin (Sigma) and Ceftazidime (Sigma), and the analgesic Ketorolac (Sigma).

There are two types of electrospinning films, BMP-2 coaxial film and CTGF coaxial film. PLGA503 and HFIP were fully mixed according to the proportions in Table 1 and Table 2, and placed on a magnetic stirrer for stirring until fully mixed as a coaxial electrospinning shell material solution. Bovine serum albumin (BSA) and BMP-2 were dissolved in phosphate buffered saline (PBS) according to the ratios described in Table 1. CTGF was dissolved in PBS according to the ratio described in Table 2, as the coaxial electrospinning core material.

TABLE 1
Preparation of coaxial thin film solutions

| | | |
|---|---|---|
| Outer layer | PLGA(503) | HFIP |
| | 840 mg | 3 ml |

| | | | |
|---|---|---|---|
| Inner layer | PBS | BMP-2 | BSA |
| | 1 ml | 20 μg | 1 μl |

TABLE 2
Preparation of CTGF coaxial thin film solutions

| | | |
|---|---|---|
| Outer layer | PLGA(503) | HFIP |

| | | | |
|---|---|---|---|
| 840 | mg | 3 | ml |

| | | |
|---|---|---|
| Inner layer | PBS | CTGF |

| | | | |
|---|---|---|---|
| 1 | ml | 20 | μg |

TABLE 3
Electrospinning parameters setting

| | | | Pushing needle | | Working |
|---|---|---|---|---|---|
| | | Voltage | rate | | distance |
| Drug film | | 17,000 V | 0.84 | ml/h | 15 cm |
| Second layer | core layer | 17,000 V | 0.1 | ml/h | 15 cm |
| of BMP-2 film | shell layer | | 0.2 | ml/h | |
| Second layer | core layer | 17,000 V | 0.1 | ml/h | 15 cm |
| of CTGF film | shell layer | | 0.2 | ml/h | |

### Example 4: Mechanical Properties Test

Knuckle fatigue testing is performed at room temperature by using LLOYD INSTRUMENTS LRX, Load cell (sensitivity 106.1% load rating 2500N). The tested joint implant prepared according to the present disclosure was placed on the material testing machine platform, and the computer-driven pressure was applied to stretch downward and relax upward to simulate the extrusion of finger joints by external force. The deformations of the tested joint implant due to external force before and after the knuckle fatigue testing were compared, and whether the knuckles are broken after 10,000 times of fatigue is observed.

The printed PCL joint implants were subjected to fatigue testing at room temperature by using a material testing machine. The results show that the tested joint implants prepared according to the present disclosure have no fracture after 10,000 fatigue tests. It proves that the joint implant prepared by the above process has sufficient flexibility and strength, which is favorable for long-term use.

### Example 5: Surface Morphology Observation

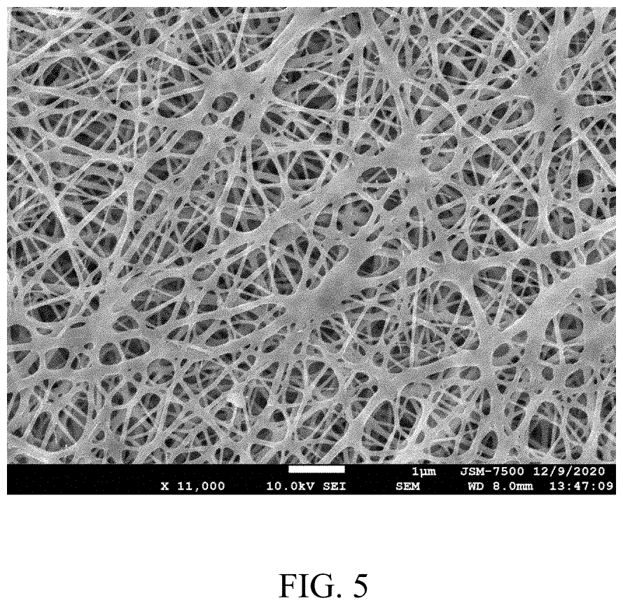

In order to confirm the morphology of the drug fiber film and calculate the fiber diameter, the surface morphology of the film was observed by using a field-emission scanning electron microscope (FE-SEM). The SEM image of the observed electrospinning fiber film is shown in FIG. 5. The figure shows that the nanofibers prepared by electrospinning technology have a high surface area-to-volume ratio, and the interconnected pores in the fibers are conducive to drug delivery, thereby ensuring higher dissolution rates and higher therapeutic drug absorption rates. Moreover, the drug release rate can be controlled for various applications by adjusting the relevant nanofiber properties, such as fiber diameter, porosity, and drug binding mechanism. In addition, the nanofiber structure may facilitate cell (such as, connective tissue cells) attachment to promote tissue healing or recovery.

### Example 6: Water Contact Angle Measurement

The water contact angle is an index of the hydrophilicity and hydrophobicity of a material. When a liquid is in contact with a solid, an angle (the angle inside the liquid) will be formed along the tangential direction of the liquid/gas interface from the triple point of the solid, liquid and gas three-phase junction. The contact angle of hydrophilic object is about 0° to 90° and the contact angle of hydrophobic object is greater than 90°. The change of the surface properties of the nanofiber film surface from hydrophobicity to hydrophilicity can be confirmed by the water contact angle. This change in the water contact angle can indicate whether the drug used is properly attached to the nanofiber film.

In this experiment, the water contact angle of electrospinning fiber film was measured by using horizontal imaging angle measurement instrument PSC-1000B (Pentad Scientific Corporation) to determine the hydrophilicity or hydrophobicity of the film.

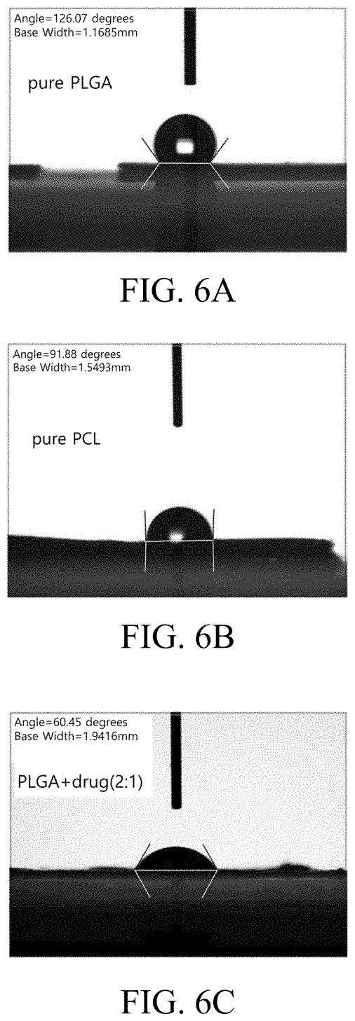

The results of the hydrophobic and hydrophilic properties of the joint implants with electrospinning fiber film carrying no drug and two drugs show that the water contact angle of the joint implant with the pure PLGA film is 126.07 degrees; the water contact angle of the joint implant with the pure PCL film is 91.88 degrees; and the water contact angle of the joint implant with the pure PLGA film carrying two drugs is 60.45 degrees (see, FIGS. 6A-6B).

### Example 7: Detection of Drug Components in the Film Layer and Detection of Long-Term Release Trends

Drug components in the electrospinning fiber film carrying the drugs were detect by using Fourier transform infrared spectrometer (FTIR SCINCO TAIWAN CO., LTD.). The used drugs, such as Ceftazidime, Teicoplanin, Ketorolec and other drugs were detected by using high performance liquid chromatography (HPLC) to verify whether the PLGA film carried drugs and to explore the long-term release trends of the drugs. The drug-loaded electrospinning fiber film carrying drugs released the drugs under a simulated environment in vitro, and then the daily releases of the drugs were detected by high performance liquid chromatography.

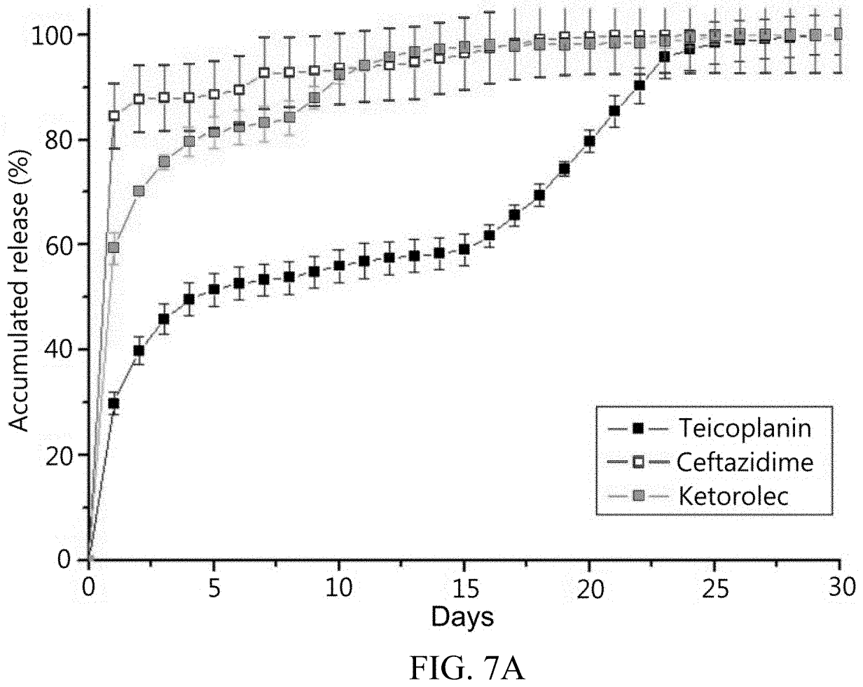

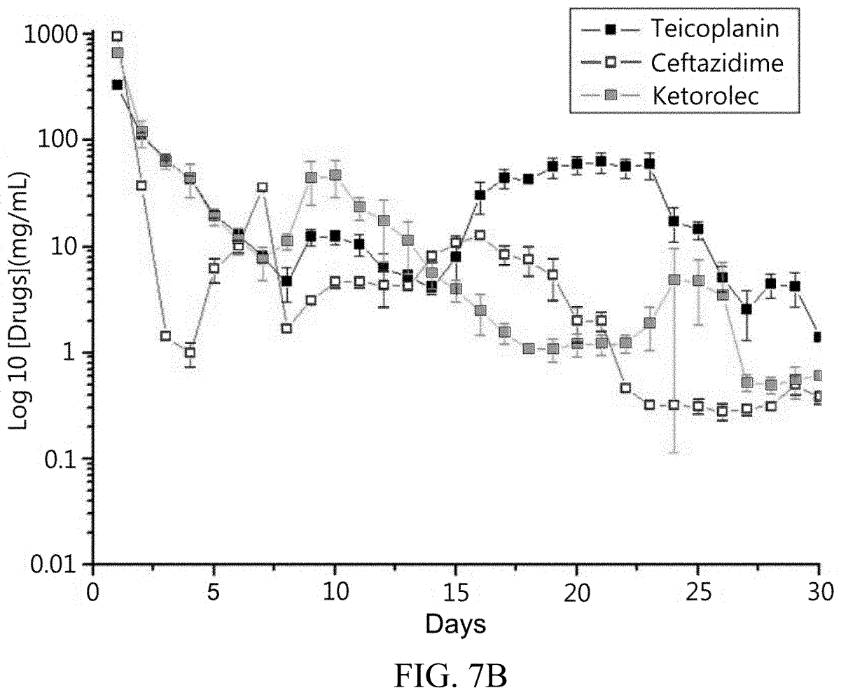

The results of long-term release of drug components in the film layer are shown in FIG. 7A and FIG. 7B. FIG. 7A shows the in vitro daily releases of drug ingredients in the film layer, and FIG. 7B shows the in vitro cumulative releases of drug ingredients in the film layer. According to the drug release trends shown in FIG. 7A and FIG. 7B, it can be demonstrated that the drug efficacy has a long-time sustained release effect.

### Example 8: In Vitro Degradation Test

The PCL scaffolds prepared according to the above 3D printing method were soaked in phosphate buffered saline (PBS) and placed in a 37° C. incubator for quantitative analysis every month. An unsoaked scaffold was used as a control group to compare the changes in material properties of the PCL scaffolds within 1 to 4 months. After the experiment, the molecular weight was quantitatively analyzed by using Gel Permeation Chromatography gel (GPC).

Gel permeation chromatography (GPC) uses the diffusion of solid particles in a fluid as the basis for separation. First, 15 mg of the PLC scaffold was placed in 1 ml of tetrahydrofuran (THF) to completely melt the PLC scaffold. Using a GPC instrument (Waters 2414, Waters 1515) and software (Breezes), the analyte solution was injected into the instrument at 40° C. for analysis to obtain values of Mz (z-average molecular weight), Mz+1 (z+1 average molecular weight), Mw (weight average molecular weight), Mn (number average molecular weight) and My (viscosity average molecular weight).

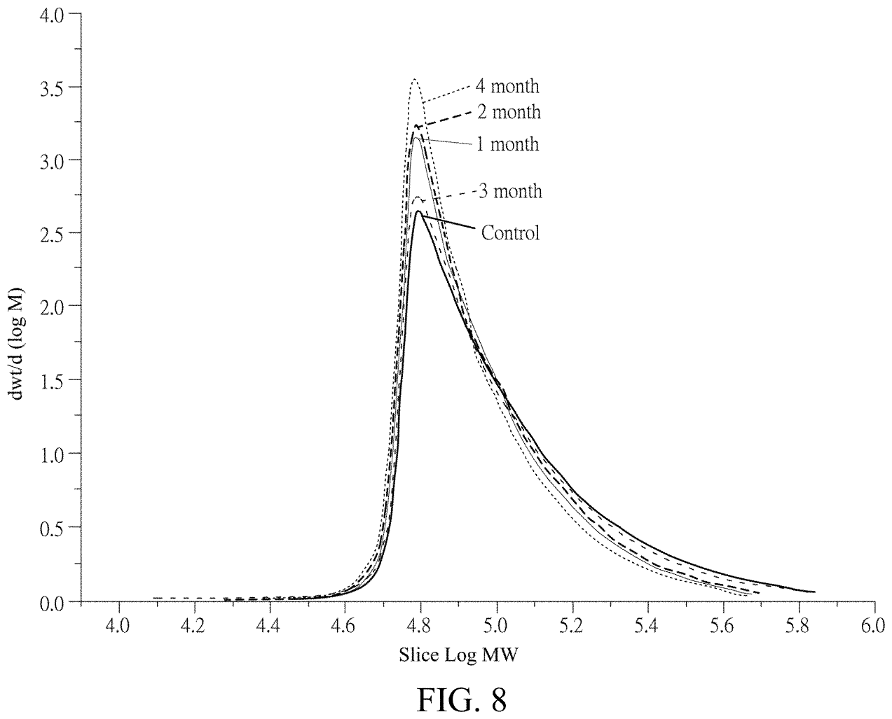

The results are shown in the following table and FIG. 8. According to the data shown in the table below and the degradation curve of the PCL scaffold shown in FIG. 8, it can be confirmed that the PLC scaffold of the present disclosure can be continuously degraded in a phosphate buffered saline (PBS) compared with the control.

TABLE 4
The compatible of degradation amount of PCL scaffold

| | Mn | Mw | Mz | Mz + 1 |
|---|---|---|---|---|
| Control | 91009 | 129339 | 253320 | 572523 |
| 1^{st} month | 88896 | 123047 | 225471 | 472372 |
| 2^{nd} month | 84217 | 108898 | 172705 | 314241 |
| 3^{rd} month | 82043 | 104272 | 157608 | 271521 |
| 4^{th} month | 79477 | 99014 | 146111 | 251662 |

### Enumerated Embodiments

The following exemplary embodiments are provided, the numbering of which is not to be construed as designating levels of importance.

Embodiment 1 provides a biodegradable joint implant, comprising
a joint scaffold comprising
   a first anchor fixing;
   a second anchor fixing; and
   a flexible spacer, wherein the first anchor fixing and the second anchor fixing are respectively axially connected to opposite sides of the flexible spacer; and
   a film layer coated on a surface of the joint scaffold,
   wherein the joint implant is made of biodegradable material, and the film layer comprises at least one substance for inducing tissue growth.

Embodiment 2 provides the joint implant of Embodiment 1, wherein the flexible spacer further comprises a longitudinal first side wall and a longitudinal second side wall, and the first anchor fixing extends laterally and outwardly from an outer side of the first side wall, the second anchor fixing extends laterally and outwardly from an outer side of the second side wall, and the first side wall and the second side wall are connected by one or more lateral connecting parts.

Embodiment 3 provides the joint implant of Embodiment 2, wherein both ends of the connecting part are respectively laterally connected to inside central portions of the first side wall and the second side wall to form an H-shaped flexible spacer.

Embodiment 4 provides the joint implant of Embodiment 2, wherein both ends of the connecting part are respectively laterally connected to inside bottoms or tops of the first side wall and the second side wall to form an inverted U-shaped or U-shaped flexible spacer.

Embodiment 5 provides the joint implant of Embodiment 2, wherein two of the connecting parts are laterally connected to the bottom and top of the first side wall respectively, each end of the two connecting parts away from the first side wall has a hole, the top and bottom of the second side wall are configured with shaft devices corresponding to the holes on the two connecting parts, the two shaft devices of the second side wall are respectively mounted into the corresponding holes on the two connecting parts to form a flexible spacer with a hinge configuration.

Embodiment 6 provides the joint implant of Embodiment 1, wherein the axial section of the joint scaffold is curved in an arc shape.

Embodiment 7 provides the joint implant of Embodiment 1, wherein a length of the flexible spacer is 10-30 mm, preferably 10-25 mm, and more preferably 10-20 mm, and can be 10, 15, 20 or 25 mm.

Embodiment 8 provides the joint implant of Embodiment 1, wherein the length of the first anchor fixing is identical with or different from that of the second anchor fixing.

Embodiment 9 provides the joint implant of Embodiment 1, wherein a length of each of the first anchor fixing and the second anchor fixing is 20-40 mm, preferably 20-35 mm, and more preferably 20-30 mm, and can be 20, 25, 30, 35 or 40 mm.

Embodiment 10 provides the joint implant of Embodiment 1, where the film layer is a nanofiber film.

Embodiment 11 provides the joint implant of Embodiment 1, wherein the biodegradable material is selected from a group consisting of poly(lactide-co-glycolide) (PLGA), polycaprolactone (PCL), and a combination thereof.

Embodiment 12 provides the joint implant of Embodiment 1, wherein the substance for inducing tissue growth is selected from a group consisting of bone morphogenetic protein 2 (BMP-2) and connective tissue growth factor (CTGF).

Embodiment 13 provides the joint implant of Embodiment 1, wherein the film layer further comprises one or more medicaments selected from a group consisting of anti-inflammatory drug, antibiotic, analgesic, and any combination thereof.

Embodiment 14 provides the joint implant of Embodiment 13, wherein the anti-inflammatory drug is selected from a group consisting of Adalimumab, Certolizumab, Etanercept, Golimumab, Abatacept, Tocilizumab, Rituximab, Infliximab, and any combination thereof.

Embodiment 15 provides the joint implant of Embodiment 13, wherein antibiotic is selected from a group consisting of Vancomycin, Teicoplanin, Ceftazidime, Gentamicin, Mezlocillin, Cloxacillin, Meticillin, Cephalothin, Lincomycin, Polymyxin E, Bacitracin, Fusidic Acid, and any combination thereof.

Embodiment 16 provides the joint implant of Embodiment 13, wherein the analgesic is selected from a group consisting of acetaminophen, Ketorolec, clonidine, benzodiazepine, lidocaine, tramadol, carbamazepine, meperidine, zaleplon, trimipramine maleate, buprenorphine, nalbuphine, pentazocain, fentanyl, propoxyphene, hydromorphone, methadone, morphine, levorphanol, hydrocodone, and any combination thereof.

Embodiment 17 provides the joint implant of Embodiment 1, wherein the joint implant is a finger joint implant or a toe joint implant.

Embodiment 18 provides a method for preparing a joint implant, comprising preparing a joint scaffold with a biodegradable material, wherein the joint scaffold comprises
a first anchor fixing;
a second anchor fixing; and
a flexible spacer, wherein the first anchor fixing and the second anchor fixing are respectively axially connected to opposite sides of the flexible spacer,
forming a film layer on the joint scaffold.

Embodiment 19 provides the method of Embodiment 18, wherein the joint scaffold is prepared by 3D bioprinting.

Embodiment 20 provides the method of Embodiment 19, wherein the 3D bioprinting is an extrusion-based bioprinting, an inkjet bioprinting or a laser-based bioprinting.

Embodiment 21 provides the method of Embodiment 18, wherein the film layer is a nanofiber film.

Embodiment 22 provides the method of Embodiment 18, wherein the film layer is prepared by an electrospinning technology.

Embodiment 23 provides a joint replacement method, comprises replacing the injured joint with the joint implant of any one of Embodiments 1-17 by performing joint replacement surgery.

Embodiment 24 provides the joint replacement method of Embodiment 23, wherein the joint is a finger joint or a toe joint.

Embodiment 25 provides the joint replacement method of Embodiment 23, wherein the injured joint is caused by rheumatoid arthritis.

Embodiment 26 provides a method for treating joint injury caused by rheumatoid arthritis, the method comprises the replacement of the injured joint with the joint implant of any of Embodiments 1-17.

Embodiment 27 provides the method of Embodiment 26, wherein the joint is a finger joint or a toe joint.

The exemplary embodiments disclosed above are merely intended to illustrate the various utilities of this disclosure. It is understood that numerous modifications, variations and combinations of functional elements and features of the present disclosure are possible in light of the above teachings and, therefore, within the scope of the appended claims, the present disclosure may be practiced otherwise than as particularly disclosed and the principles of this disclosure can be extended easily with appropriate modifications to other applications.

All patents and publications are herein incorporated for reference to the same extent as if each individual publication was specifically and individually indicated to be incorporated by reference. It should be understood that although the present disclosure has been specifically disclosed by preferred embodiments and optional features, modification and variation of the concepts herein disclosed may be resorted by those skilled in the art, and that such modifications and variations are considered to be within the scope of this disclosure.

### REFERENCES

1 Swanson A B., “Flexible implant arthroplasty for arthritic finger joints. Rational, technique and results of treatment,” J Bone Joint Surg., 1972.
2. I. A. Trail et al., “Seventeen-year survivorship analysis of silastic metacarpophalangeal joint replacement,” The Bone & Joint Journal., 2004.
3. Yajie Zhong, Patrick Godwin, Yongcan Jin, Huining Xiao, “Biodegradable polymers and green-based antimicrobial packaging materials: A mini-review,” Advanced Industrial and Engineering Polymer Research, pp. 27-35, 2020.
4. Hirenkumar K. Makadia, Steven J. Siegel, “Poly Lactic-co-Glycolic Acid (PLGA) as Biodegradable Controlled Drug Delivery Carrier,” Bioinspired Polymers, pp. 1377-1397, 2011.
5. Zheng-MingHuang, Y.-Z.ZhangbM.KotakicS.Ramakrishna, “A review on polymer nanofibers by electrospinning and their applications in nanocomposites,” Composites Science and Technology, Pages 2223-2253, 2003.
6. Di Chen, Ming Zhao, Gregory R. Mundy, “Bone Morphogenetic Proteins,” Growth Factors, pp. 233-241, 2004.
7 Stephan Barrientos, Olivera Stojadinovic, Michael S. Golinko, Harold Brem, Marjana Tomic-Canic,” Growth factors and cytokines in wound healing,” Wound Repair and Regeneration, pp. 585-601, 2008.
8. Wei Zhu, Xuanyi Ma, Maling Gou, Deqing Mei, Kang Zhang, Shaochen Chen, “3D printing of functional biomaterials for tissue engineering,” Current Opinion in Biotechnology, pp. 103-112, 2016.
9. Christian Mandrycky, Zongjie Wang, Keekyoung Kimb, Deok-Ho Kim, “3D bioprinting for engineering complex tissues,” Biotechnology Advances, pp. 422-434, 2016.

1. A biodegradable joint implant, comprising:
a joint scaffold comprising
a first anchor fixing,
a second anchor fixing, and
a flexible spacer, wherein the first anchor fixing and the second anchor fixing are respectively axially connected to opposite sides of the flexible spacer; and
a film layer coated on a surface of the joint scaffold,
wherein the joint implant is made of biodegradable material, and the film layer comprises at least one substance for inducing tissue growth.

2. The joint implant of claim 1, wherein the flexible spacer further comprises a longitudinal first side wall and a longitudinal second side wall, and the first anchor fixing extends laterally and outwardly from an outer side of the first side wall, the second anchor fixing extends laterally and outwardly from an outer side of the second side wall, and the first side wall and the second side wall are connected by one or more lateral connecting parts.

3. The joint implant of claim 2, wherein both ends of the connecting part are respectively laterally connected to inside central portions of the first side wall and the second side wall to form an H-shaped flexible spacer.

4. The joint implant of claim 2, wherein both ends of the connecting part are respectively laterally connected to inside bottoms or tops of the first side wall and the second side wall to form an inverted U-shaped or U-shaped flexible spacer.

5. The joint implant of claim 2, wherein two of the connecting parts are laterally connected to the bottom and top of the first side wall respectively, each end of the two connecting parts away from the first side wall has a hole, the top and bottom of the second side wall are configured with shaft devices corresponding to the holes on the two connecting parts, the two shaft devices of the second side wall are respectively mounted into the corresponding holes on the two connecting parts to form a flexible spacer with a hinge configuration.

6. The joint implant of claim 1, wherein a length of the flexible spacer is 10-30 mm.

7. The joint implant of claim 1, wherein a length of each of the first anchor fixing and the second anchor fixing is 20-40 mm.

8. The joint implant of claim 1, wherein the film layer is a nanofiber film.

9. The joint implant of claim 1, wherein the biodegradable material is selected from a group consisting of poly(lactide-co-glycolide) (PLGA), polycaprolactone (PCL), and a combination thereof.

10. The joint implant of claim 1, wherein the substance for inducing tissue growth is selected from a group consisting of bone morphogenetic protein 2 (BMP-2) and connective tissue growth factor (CTGF).

11. The joint implant of claim 1, wherein the film layer further comprises an anti-inflammatory drug selected from a group consisting of Adalimumab, Certolizumab, Etanercept, Golimumab, Abatacept, Tocilizumab, Rituximab, Infliximab, and any combination thereof.

12. The joint implant of claim 1, wherein the film layer further comprises an antibiotic selected from a group consisting of Vancomycin, Teicoplanin, Ceftazidime, Gentamicin, Mezlocillin, Cloxacillin, Meticillin, Cephalothin, Lincomycin, Polymyxin E, Bacitracin, Fusidic Acid, and any combination thereof.

13. The joint implant of claim 1, wherein the film layer further comprises an analgesic selected from a group consisting of acetaminophen, Ketorolec, clonidine, benzodiazepine, lidocaine, tramadol, carbamazepine, meperidine, zaleplon, trimipramine maleate, buprenorphine, nalbuphine, pentazocain, fentanyl, propoxyphene, hydromorphone, methadone, morphine, levorphanol, hydrocodone, and any combination thereof.

14. The joint implant of claim 1, wherein the joint implant is a finger joint implant or a toe joint implant.

15. A method for preparing a joint implant, comprising:
preparing a joint scaffold with a biodegradable material, wherein the joint scaffold comprises
a first anchor fixing,
a second anchor fixing, and
a flexible spacer, wherein the first anchor fixing and the second anchor fixing are respectively axially connected to opposite sides of the flexible spacer,
forming a film layer on the joint scaffold.

16. The method of claim 15, wherein the joint scaffold is prepared by extrusion-based 3D bioprinting, inkjet 3D bioprinting or laser-based 3D bioprinting.

17. The method of claim 15, wherein the film layer is a nanofiber film prepared by an electrospinning technology.

18. A joint replacement method, comprises replacing the injured joint with the joint implant of claim 1 by performing joint replacement surgery.

19. The joint replacement method of claim 18, wherein the injured joint is a finger joint or a toe joint.

20. The joint replacement method of claim 18, wherein the injured joint is caused by rheumatoid arthritis.
